(19) United States
(12) Patent Application Publication
(10) Pub. No.: US 2005/0191269 A1
(43) Pub. Date: Sep. 1, 2005

(54) Automatic air fragrance unit

(76) Inventor: Anton Dewayne Glassco, North Little Rock, AR (US)

Correspondence Address: Anton Dewayne Glassco, 701 West 18th Apt # 124, North Little Rock, AR, 72114, US

(21) Appl. No.: 11/002,406

(22) Filed: Feb. 25, 2005

Related U.S. Application Data
(60) Provisional application No. 60/528,234, filed on Dec. 9, 2003.

(51) Int. Cl. A61L 9/01

(52) U.S. Cl. 424/76.1; 55/338

(57) ABSTRACT

A new automatic air fragrance unit for radiating a pleasant scent into vehicles, homes and businesses. This unit is made of metal, plastic, rubber and steel.

This invention relates to a built in fragrance unit and more particularly to an arrangement for selectively refreshing the air within the passenger compartment of a vehicle, home and businesses.

Devices for deodorizing the air, and for producing a pleasant aroma, within a vehicle area available in a variety of arrangements and configurations, as seen in several United States Patents.

Suzuki et al (U.S. Pat. No. 4,345,550) discloses an air filtering system, which is not conveniently located and is excessively large and complicated. Shields et al (U.S. Pat. No. 2,778,678) is for a deodorant dispenser, which is excessively complicated and expensive. Sone et al (U.S. Pat. No. 4,274,843) disclose electrostatic air purifier, which does not for an addition of a fragrance to the air in an automobile.

Miller (U.S. Pat. No. 4,309,382) shows a complicated and expensive apparatus for deodorizing the air while Gilbertson (U.S. Pat. No. 3,722,182) shows a complicated and clumsy device for deodorizing the air in automobiles. Also, it does not provide for the addition for a fragrance.

Spector (U.S. Pat. No. 4,523,670) acts like a hanging air freshener and could be inconvenient and interfere with activities in the driving compartment. The patent to Baker et al (U.S. Pat. No. 2,731,104) only provides for inaccessible location.

The patent to Deguisseppe (U.S. Pat. No. 4,722,264) is a aroma producing apparatus, which is not conveniently located and could interfere with the operation of the driving compartment while Lorman et al (U.S. Pat. No. 5,853,672) shows an air dispensing system, which could be expensive and inconvenient. Also, the light displayed occasionally could be distracting.

It is seen that these patented arrangements are either complicated or therefore expensive to install, inefficient, or are highly inconvenient to use.

### SUMMARY OF PRESENT INVENTION

In this invention there is provided an arrangement for deodorizing or imparting a pleasant odor to the air within a vehicle, home or business, which is simple, economic, highly efficient, and very effective.

In accordance with a preferred embodiment of this invention there is provided a housing which may be mounted within a truck or under the hood of a vehicle or any where in a home or business having a polyethylene tubing and a reducing coupling and a spray mist nozzle. Also having push button switch or remote and also preprogrammed in the controller to active the system. The controller has FBD functions 21 preprogrammed functions for counting, time, comparison, multiplexing, time-based programming and display. The FBD functions are the following Boolean-used to create logic equations between connected inputs. Set-reset-Bistable memory-priority assigned to either set or reset. Time A/C-time-a/c function (Delay on/Delay off). Up down count, preset count-selection up/down counter. Comp in zone-zone comparison (Min<value<max). Time BW-timer-BW function (Pulse on a rising or falling edge). Preset H-Meter-hour counter (selection of hour, minute). Timer Li-pulse generator (on setting, off setting). Timer B/H-function (Adjustable pulsed signal). Trigger-Defines an activation zone with Hysteresis. Compare-Comparison of 2 analogue values using the =,>,<, operators Gain used to convert an analogue value by changing the scale and offset. Display—Of digital and analogue data, date, time, messages for man-machine interface. Bistable—Impulse relay function. Time program—daily, weekly, and yearly time programmer. MUX-multiplexing functions on 2 analogue values. PID—Temperature controller. Store—storage of data values with an average value, H-Speed Count-High-speed counting 1 kHz for up-down, 500 Hz for Phase tachometer function as standard. Timer-4 sub-functions At, Ht, T which add up the total opening or closing time of contact. Tt: timed impulse relay. Logic functions and, OR, NAND, NOR, XOR, NOT functions. Outputs-you have two types of output: physical (digital, solid state, PWM) or internal (backlighting). Input-you can connect digital, analogue or potentiometer physical inputs you also have internal inputs such as keypad, constants, etc. SFC functions (GRAFCET) All the GRAFCET functions have been integrated with either backup or reset in the event of mains power failure (step, transition, divergence, convergence, etc). The controller opens and closes the valves so that gravity can pull the fragrance out of the bottle to the valves also open and close so that the compress air will push the fragrance out of the valves to the spray nozzle.

The principle object of this invention is to provide an improved, economic, convenient, and practical way to fragrant the air in the compartment of a vehicle. Also, imparting fragrance into the air in the home and businesses.

Other objects and advantages of this invention will hereinafter become obvious from the following description of preferred embodiments of this invention.

### BRIEF DESCRIPTION OF THE DRAWING

FIG. d1. Is a side top view of the box operator controller.

FIG. d2. Is a side top view of the cover box operator controller.

FIG. d3. Is a side top view of the sides and back of unit.

FIG. d4. Is a side front top view of the front of unit.

FIG. d5. Is a side top view of the bottom cover tray of unit.

FIG. d6. Is a side top view of the tray top of unit.

FIG. d7. Is a side top view of the tray bottom of unit.

FIG. d8. Is a side top view of the top cover of unit.

FIG. d9. Is a side top view of the instant tube fitting inside unit.

FIG. d10. Is a side top view of the bottles inside the unit.

FIG. d11. Is a side top view of the valves inside the unit.

FIG. d12. Is a side top view of the controller inside the unit.

FIG. d13. Is a side top view of the tube pipe connector inside the unit.

FIG. d14. Is a side top view of the air compressor inside the unit.

FIG. d15. Is a side top view of the screw.

FIG. d16. Is a side top view of the straight fitting tube.

FIG. d17. Is a side top view of the streetel.

FIG. d18. Is a side top view of the air line connector.

FIG. d19. Is a side top view of the push button switch.

FIG. d20. Is a side top view of the polyethylene tubing inside the unit.

FIG. d21. Is a side top view of the spray mist nozzle.

FIG. d22. Is a side top view of the reducing coupling.

FIG. d23. Is a side top view of the polyethylene tubing outside the unit.

FIG. d24. Is a side top view of the cord grip outside the unit.

FIG. d25. Is a side top view of the 12 or 220 volt ext. cord outside the unit.

FIG. d26. Is a side top view of the relay and relay socket inside the unit.

FIG. d27. Is a side top view of the nipple inside the unit.

FIG. d28. Is a side top view of the mnpt tube to pipe adapter.

FIG. a29. Is a side view of the air flow tubing

FIG. a30. Is a top view of the fan and filter housing

FIG. a31. Is a top view of the fan with blades.

FIG. a32. Is a top view of the filter.

FIG. a33. Is a top view of the vent cover.

DRAWING. A. Is a side view of the vent cover, filter, fan with blades and vent, filter, fan, housing.

DRAWING. B. Is a side view of the vent cover, filter, air chamber, spray mist nozzle and the air flow tubing connection.

DRAWING. C. Is a side view of the Clean Smell System.

DRAWING. D. Is a side top view of the Clean Smell unit.

### DETAILED DESCRIPTION OF THE PREFERRED EMBODIMENTS

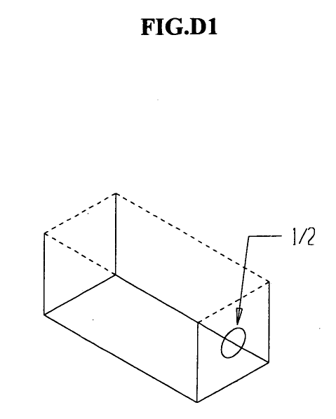

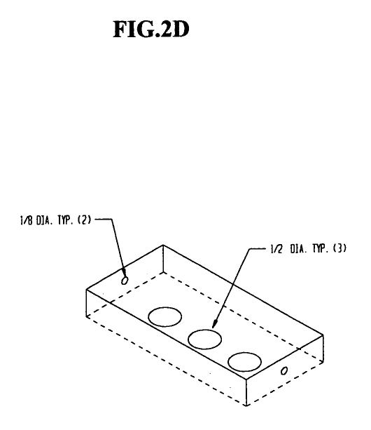

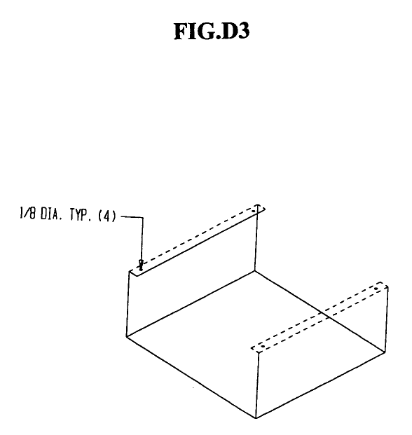

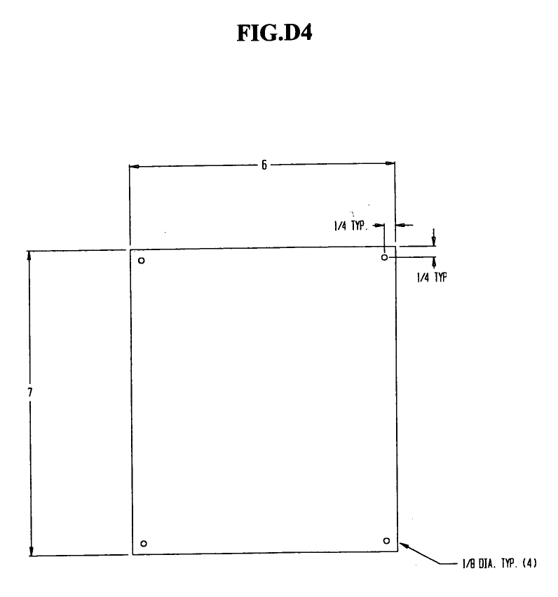

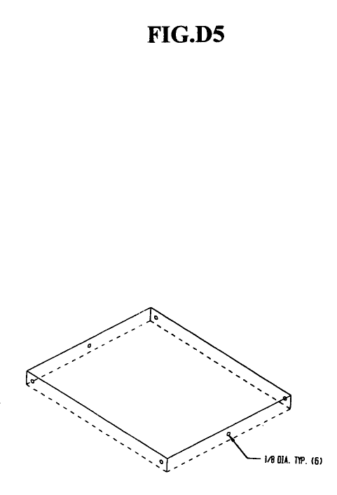

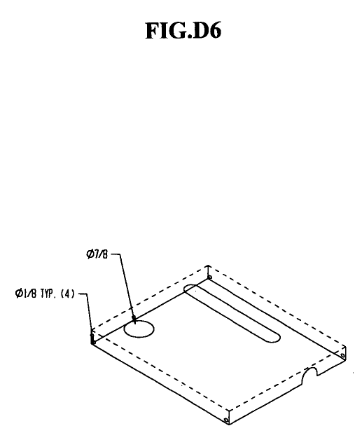

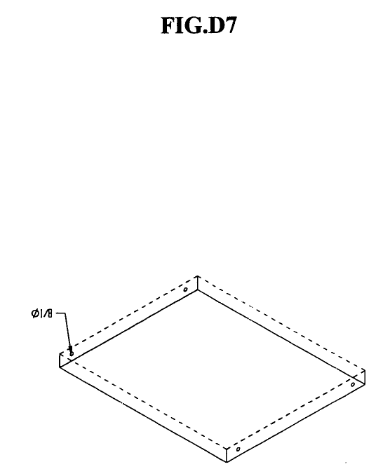

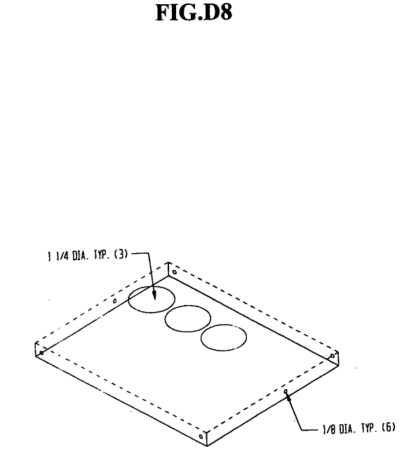

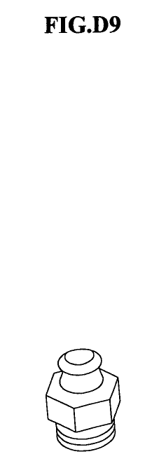

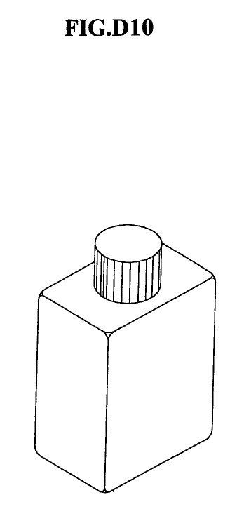

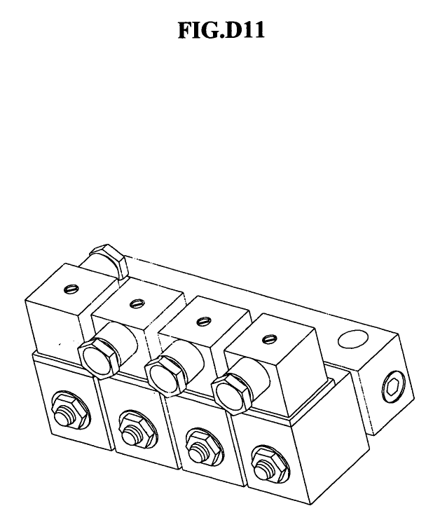

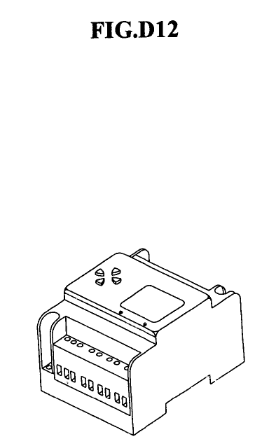

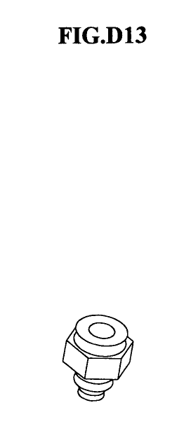

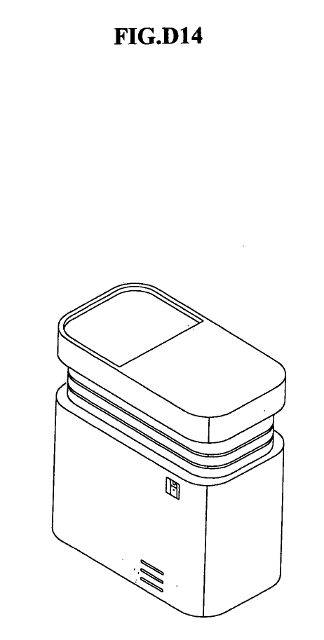

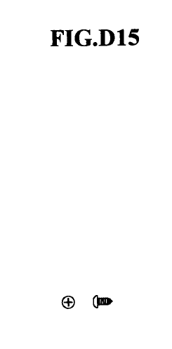

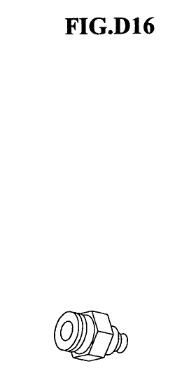

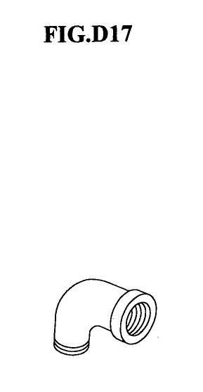

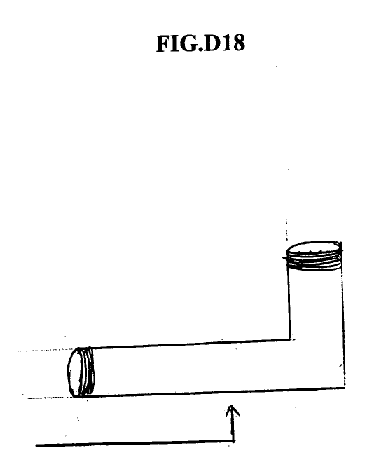

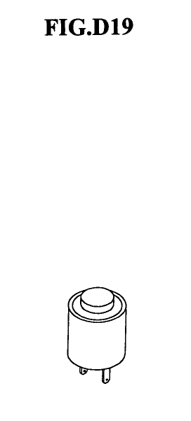

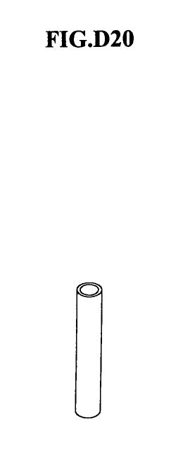

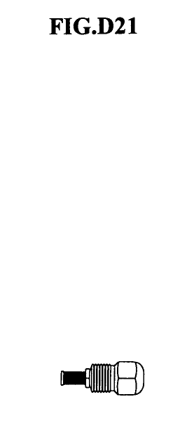

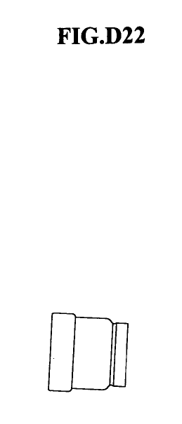

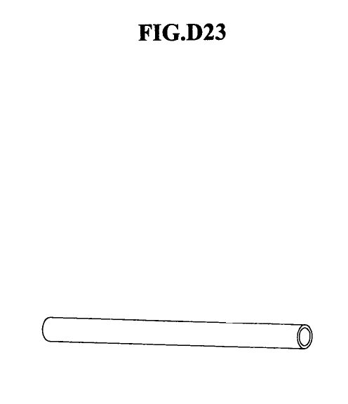

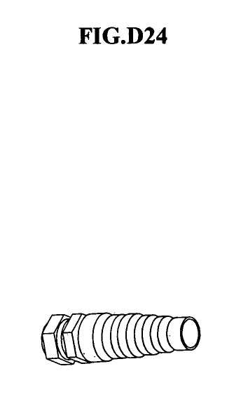

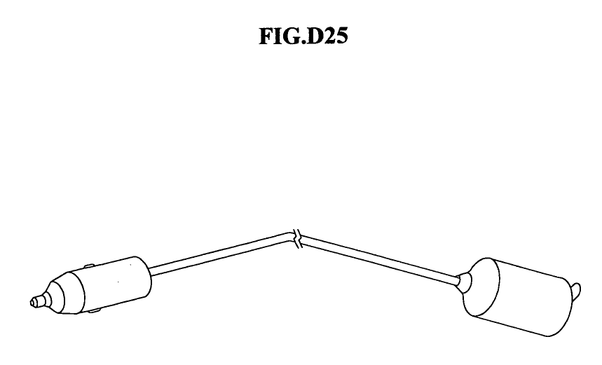

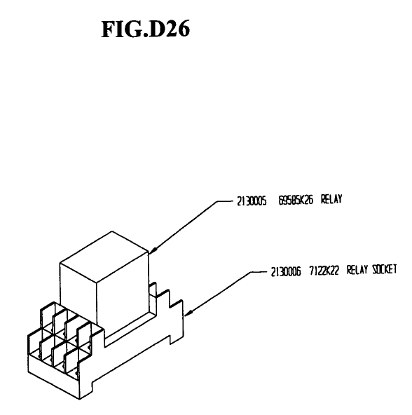

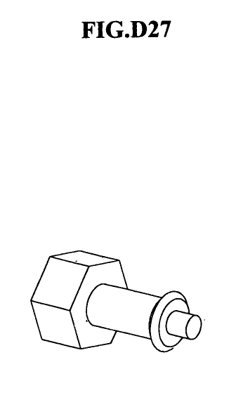

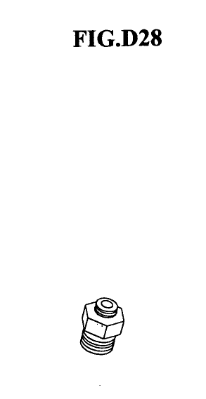

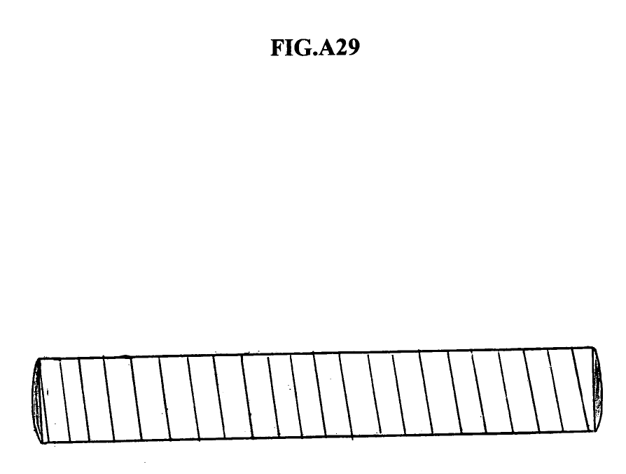

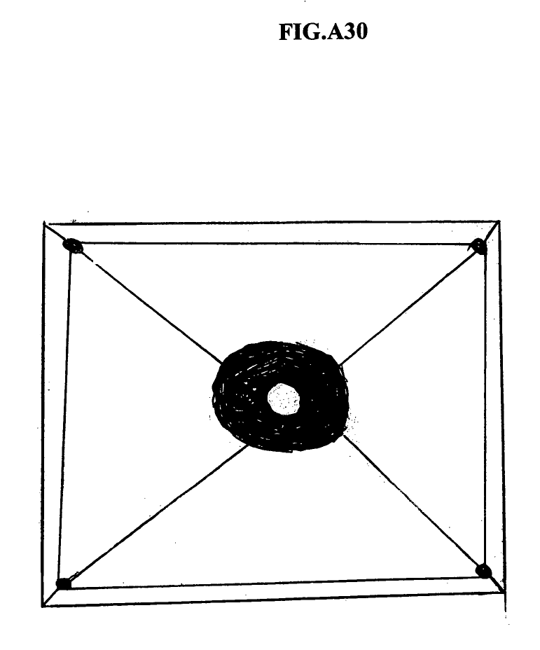

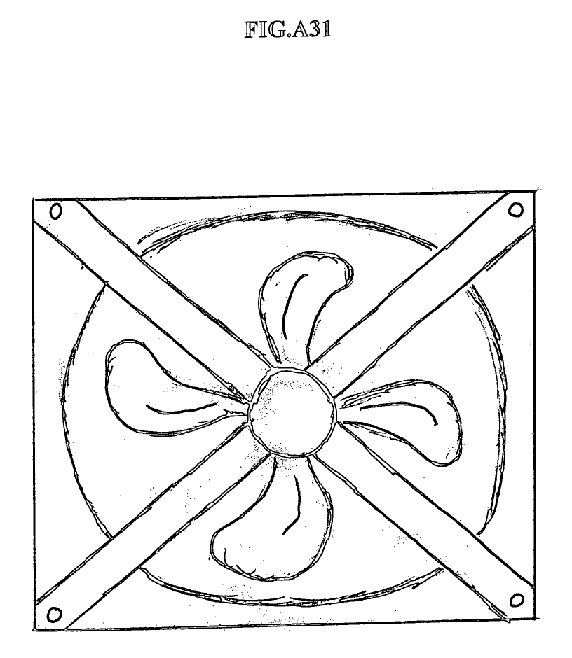

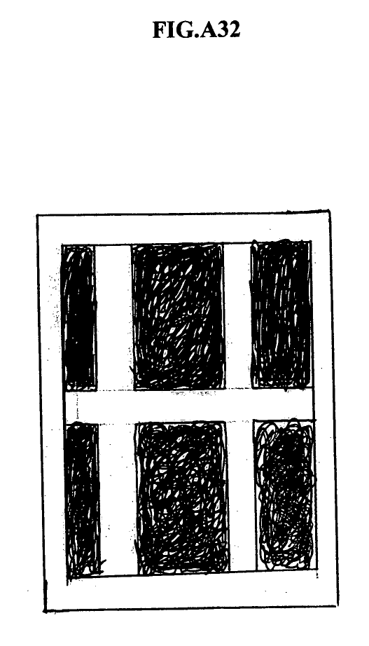

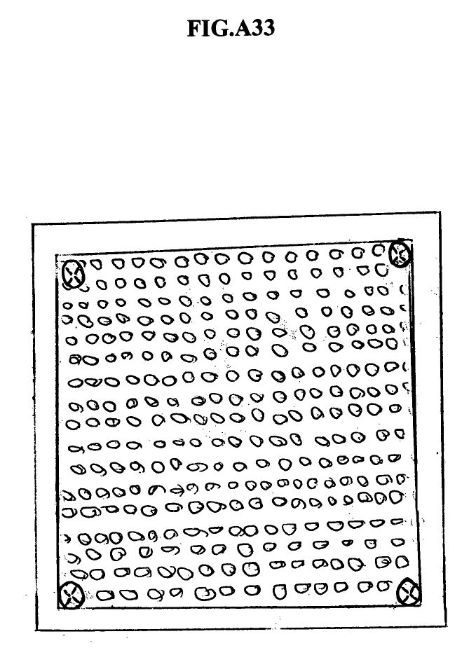

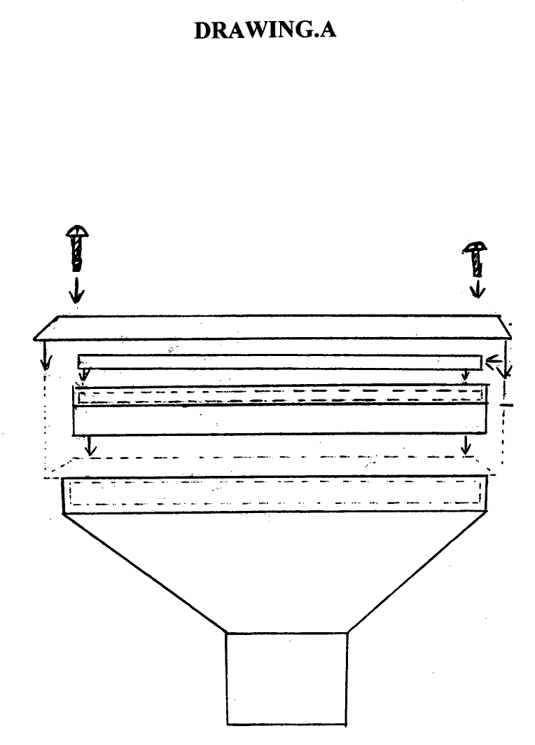

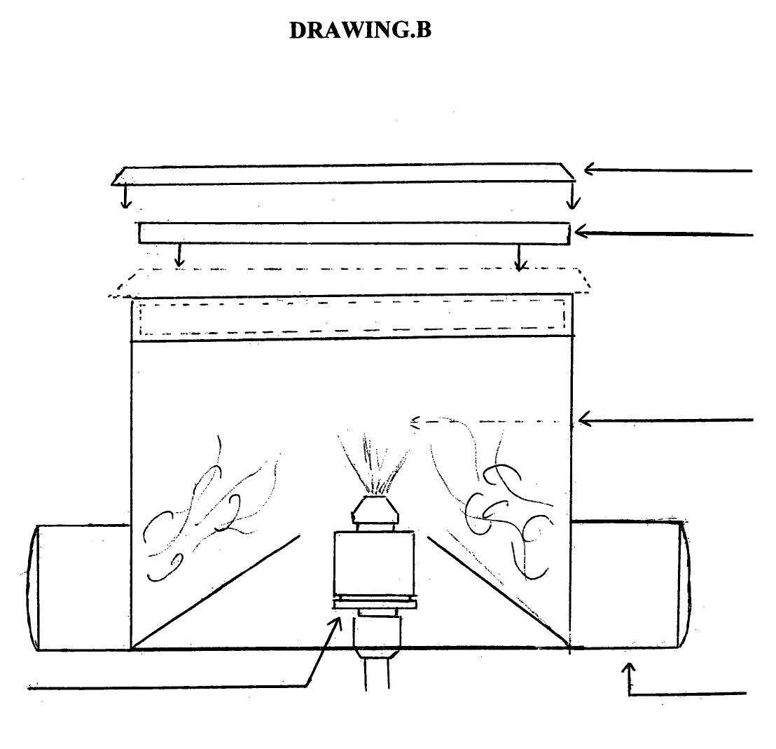

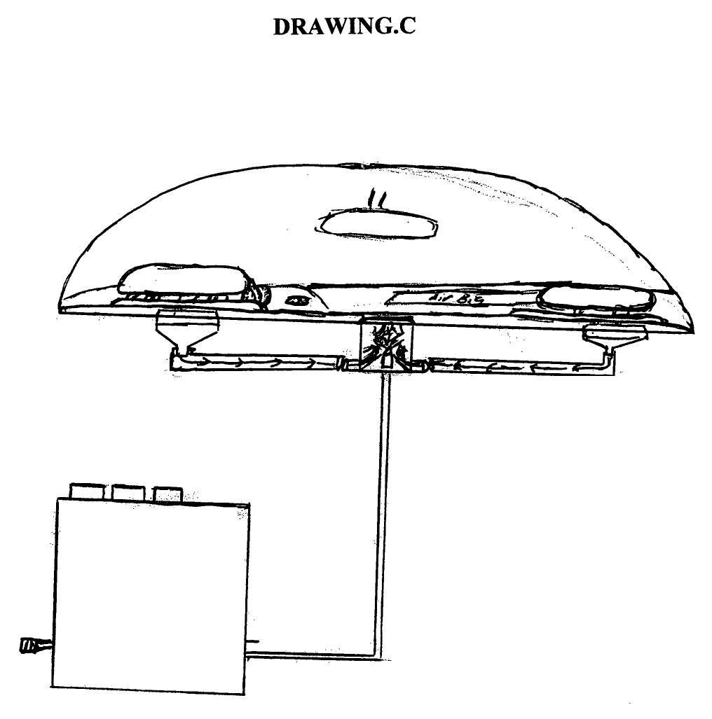

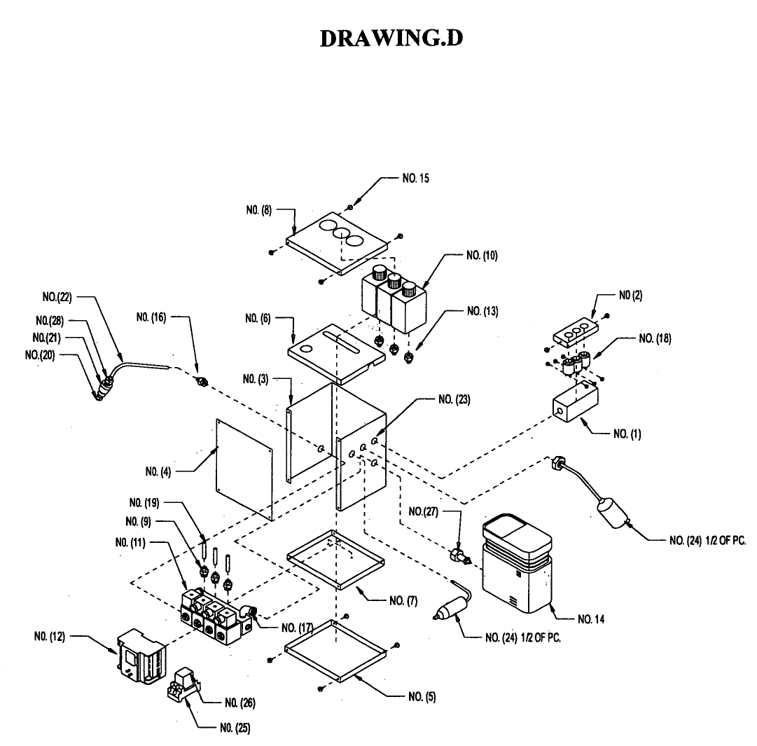

FIG. d12. Is the controller, it controls the amount of fragrance that is pulled down out of FIG. d10. 1 of 3 bottles and down pass FIG. d13. 1 of 3 tubing pipe connections. Down pass FIG. d19. 1 of 3 Polyethylene tubing down pass FIG. 9. Instant tube fitting down pass into FIG. d11. Vales where the compress air and the fragrance mix and push out of the vales and out pass FIG. d16. 51025 k straight fitting tube into FIG. d22. Polyethylene tubing into FIG. d28. MNPT tube to pipe adapter into FIG. d21. Reducing coupling fitting and than the compress air and fragrance out of FIG. d20. Spray mist nozzle. FIG. d26. Relay and relay socket and having wires than connect to FIG. d12. the controller FIG. d18. 3 push button system and having wires than connect to FIG. d12. the controller and the controller than signals FIG. d14. The air compressor to release compress air the air than pass FIG. d27. The nipple than the air pass into FIG. d17. The streetel and connected to it is FIG. d18. The air filler pipe the air than pass to air filling tool or pass to FIG. d11. The valves and having wires connecting to FIG. d12. The controller.

FIG. d25. The 12 or 220 volt ext cord from the controller to a 12 or 220 volt electrical system.

FIG. d5. The bottom cover holds FIG. d7. The bottom tray and the FIG. d11, d12, d25, d26, is mounted to FIG. d7. the bottom tray FIG. d3. The side and back cover fit down into FIG. d5. the bottom cover FIG. d6. is mounted in the middle of FIG. d3. the side bottom and back cover FIG. d4. the front cover connects to FIG. d5, d7, d3, and FIG. d8. the top cover connects to FIG. d3, d4,. And FIG. d20, d21, d28, d22, connects to DRAWING. B. the air chamber and FIG. a32, a33, fits into DRAWING. B. and FIG. a29. air flow tubing connects to DRAWING. B. the air flow chamber.

FIG. a29. the air flow tubing connects to DRAWING. A. The vent, filter and fan housing FIG. a31. The fan with blades fits into DRAWING. A. The filter and fan housing and FIG. a32. The filter fits over FIG. a31. the fan with blades and FIG. a33. The vent cover fits over FIG. a31, a32 DRAWING A, the vent, filter and fan housing.

FIG. d12. The controller has 21 programmed functions. For counting, timing, comparison, multiplexing, time based programming and display. The FBD functions are the following Boolean-used to create logic equations between connected inputs. Set-reset-Bistable memory-priority assigned to either set or reset. Time A/C-time-a/c function (Delay on/Delay off). Up down count, preset count—selection up/down counter. Comp in zone—zone comparison (MIN<value<max). Timer BW—timer—BW function (Pulse on a rising or falling edge). Preset H-Meter—hour counter (selection of hour, minute). Timer LI—pulse generator (on setting, off setting). Timer B/H—timer B/H function (adjustable pulsed signal). Trigger—Defines an activation zone with Hysteresis. Compare—Comparison of 2 analogue values using the =, >, <, operator. Gain—used to convert an analogue value by changing the scale and offset display—of digital and analogue data, date, time, messages for man—machine interface. Bistable—Impulse relay function. Time program—daily, weekly and yearly time programmer. MUX-multiplexing functions on 2 analogue values. PID—Temperature Controller. Store storage of data values with an average value, H-Speed Count—High speed counting 1 kHz for up-down, 500 Hz for Phase tachometer function as standard. Timer-4 sub-functions At, Ht, T which add up the total opening or closing time of contact. Tt: timed impulse relay. Logic functions and, OR, NAND, NOR, XOR, NOT functions. Outputs—you have two types of output: physical (digital, solid state, PWM) or internal (backlighting). Input—You connect digital, analogue or potentiometer physical inputs you also have internal inputs such as keypad constants etc. SFC functions (GRAFCET) All the GRAFCET functions have been intergrated with either backup or reset in the event of main power failure (step, transition, divergence, convergence, etc).

1. This unit will change from a 1 bottle unit to 1,000 bottle unit for vehicles, homes and businesses for imparting a pleasant fragrance to the air and will be the first fragrance unit that has the programming of a complete vehicle, home and business control system.

2. This has a 3 part vent and filter system that can be mounted in every room or office and any where in the vehicle giving a different scent to each vent system, also simple to operate and the controller has FBD functions and 21 preprogrammed functions that list in the DETAILED DESCRPTION OF THE PREFERRED EMBODIMENTS.

3. It also inflates tires on a vehicle when the tire connection hose is connected to the clean smell unit and the unit will connect to any electrical system and computer.
